(19) United States
(12) Patent Application Publication
(10) Pub. No.: US 2014/0350647 A1
(43) Pub. Date: Nov. 27, 2014

(54) ELECTROMAGNETIC HYPERTHERMIA ASSEMBLY

(71) Applicants: NATIONAL CHENG KUNG UNIVERSITY, Tainan City (TW); METAL INDUSTRIES RESEARCH & DEVELOPMENT CENTRE, Kaohsiung City (TW)

(72) Inventors: Yi-San CHANG, Tainan City (TW); Tsung-Chih YU, Tainan City (TW); Xi-Zhang LIN, Tainan City (TW); Sheng-Jye HWANG, Tainan City (TW); Ying-Jui CHAO, Tainan City (TW); Syuan-Fong CHEN, Kaohsiung City (TW); Guan-Cheng CHEN, Tainan City (TW); Tung-Chieh YANG, Linbian Township (TW); Shyang-Jye CHANG, New Taipei City (TW)

(73) Assignees: NATIONAL CHENG KUNG UNIVERSITY, Tainan City (TW); METAL INDUSTRIES RESEARCH & DEVELOPMENT CENTRE, Kaohsiung City (TW)

(21) Appl. No.: 14/281,480

(22) Filed: May 19, 2014

(30) Foreign Application Priority Data
May 21, 2013 (TW) 102117894

(51) Int. Cl. A61N 1/28; A61F 7/00

(52) U.S. Cl. CPC A61N 1/28; A61F 7/007
USPC 607/103

(57) ABSTRACT

An electromagnetic hyperthermia assembly includes: a substrate; a plurality of hyperthermia needles each having a needle portion that extends through and downwardly from the substrate and that has a needle tip distal from the substrate; and a guiding plate having a plurality of through holes. The needle portions of the hyperthermia needles respectively and removably extend through the through holes such that the guiding plate is movable along the needle portions and between the substrate and the needle tips of the needle portions. The through holes keep the needle tips spaced apart from each other at predetermined positions.

### CROSS-REFERENCE TO RELATED APPLICATION

This application claims priority to Taiwanese Application No. 102117894, filed on May 21, 2013.

### BACKGROUND OF THE INVENTION

1. Field of the Invention

The invention relates to an electromagnetic hyperthermia assembly, more particularly to an electromagnetic hyperthermia assembly including a plurality of hyperthermia needles and a guiding plate that keeps the hyperthermia needles spaced apart from each other.

2. Description of the Related Art

In order to improve a therapeutic effect of a treatment for benign or cancerous tumor tissue, hyperthermia therapies for killing the tumor tissue, such as radio frequency ablation (RFA) and microwave ablation (MWA), have been developed in the medical field. Although the RFA and MWA are adapted for treatments for local neoplasm, the required armamentaria are relatively expensive and unaffordable by a lot of patients.

Recently, another type of hyperthermia therapy, electromagnetic (EM) hyperthermia therapy, has been developed. The EM hyperthermia therapy involves insertion of a magnetically inducible hyperthermia device into the tumor tissue to be treated. Necrosis of the tumor tissue is caused by heat generated by a magnetically induced hyperthermia device under an induced magnetic field. The EM hyperthermia therapy gradually attracts attention because of the relatively good therapeutic effect of the EM hyperthermia therapy and the relatively low production cost of the magnetically inducible hyperthermia device.

Conventionally, there are single-needle and multi-needle type magnetically inducible hyperthermia devices. The multiple-needle type includes a plurality of needles that are oriented and secured on a flexible carrier so as to adjust the shaped defined by needle tips of the needles to comply with the contour of the tumor tissue or organ to be treated through deformation of the flexible carrier. Since the needles are secured on the flexible carrier, once the flexible carrier or one of the needles is damaged, repair is difficult and the conventional multi-needle type magnetically inducible hyperthermia device generally has to be disposed in its entirety. This results in unnecessary waste of medical resources. Moreover, since the needles are not detachable from the flexible carrier, dust or contaminants, such as bloodstains or removed tissue, are likely to accumulate in gaps formed between the needles and the flexible carrier and are difficult to clean.

### SUMMARY OF THE INVENTION

Therefore, an object of the present invention is to provide an electromagnetic hyperthermia assembly that can alleviate the aforesaid drawbacks of the prior art.

According to one aspect of this present invention, an electromagnetic hyperthermia assembly includes a substrate, a plurality of spaced-apart hyperthermia needles and a guiding plate.

Each of the hyperthermia needles has a needle portion that extends through and downwardly from the substrate and that has a needle tip distal from the substrate.

The guiding plate is disposed below the substrate and has a plurality of spaced-apart through holes. The needle portions of the hyperthermia needles respectively and removably extend through the through holes such that the guiding plate is movable along the needle portions and between the substrate and the needle tips of the needle portions, and the through holes keep the needle tips spaced apart from each other at predetermined positions.

According to another aspect of this present invention, an electromagnetic hyperthermia assembly includes a substrate, a plurality of spaced-apart hyperthermia needles, a thermometric needle and a thermometric detector.

The hyperthermia needles are respectively connected to the substrate. Each of the hyperthermia needles has a needle portion that extends through and downwardly from the substrate and that has a needle tip distal from the substrate.

The thermometric needle is attached to the substrate, and extends through and downwardly from the substrate.

The thermometric detector is disposed inside the thermometric needle.

### BRIEF DESCRIPTION OF THE DRAWINGS

Other features and advantages of the present invention will become apparent in the following detailed description of the preferred embodiments with reference to the accompanying drawings, of which:

FIG. 1 is a partly-fragmentary perspective view of the first preferred embodiment of an electromagnetic hyperthermia assembly according to the present invention;

FIG. 2 is an exploded, partly-fragmentary perspective, view of the first preferred embodiment;

FIG. 3 is a partly-fragmentary sectional view of the first preferred embodiment;

FIG. 4 is a partly-fragmentary sectional view of the second preferred embodiment of an electromagnetic hyperthermia assembly according to the present invention; and

FIG. 5 is a partly-exploded perspective view of the third preferred embodiment of an electromagnetic hyperthermia assembly according to the present invention.

### DETAILED DESCRIPTION OF THE PREFERRED EMBODIMENTS

Before the present invention is described in greater detail, it should be noted that like elements are denoted by the same reference numerals throughout the disclosure.

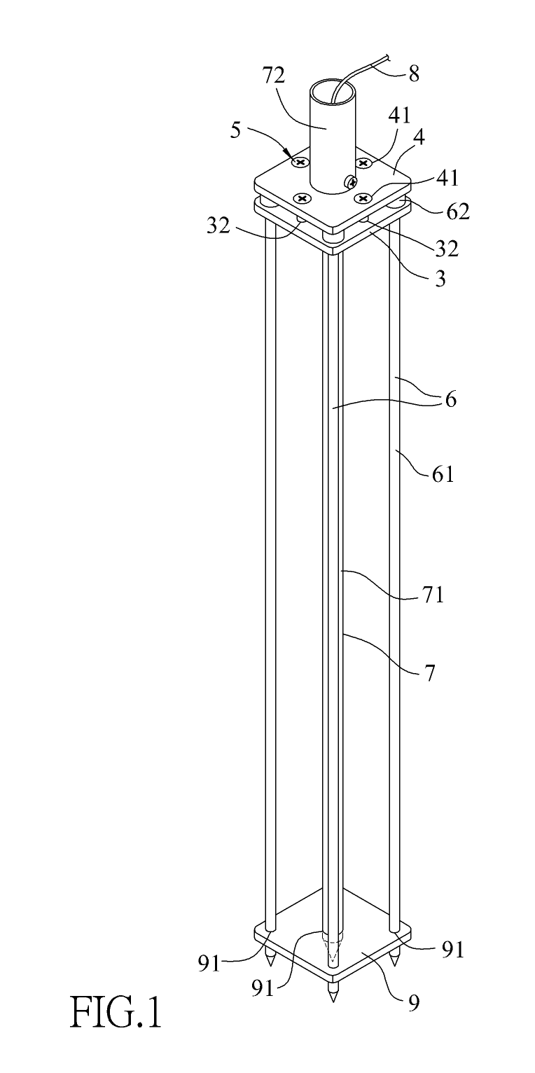

Referring to FIGS. 1 to 3, the first preferred embodiment of an electromagnetic hyperthermia assembly according to the present invention is shown. The electromagnetic hyperthermia assembly is able to inductively generate heat based on changes of a magnetic field and perform hyperthermia therapy on a tissue or organ of a living body to be treated.

In this preferred embodiment, the electromagnetic hyperthermia assembly includes a substrate 3, a plurality of spaced-apart hyperthermia needles 6 respectively connected to the substrate 3, and a guiding plate 9 that is disposed below the substrate 3 and that has a plurality of spaced-apart through holes 91. Each of the hyperthermia needles 6 has a needle portion 61 that extends through and downwardly from the substrate 3 and that has a needle tip distal from the substrate 3. The needle portions 61 of the hyperthermia needles respectively and removably extend through corresponding ones of the through holes 91 such that the guiding plate 9 is movable along the needle portions 61 between the substrate 3 and the needle tips of the needle portions 61. The through holes 91 keep the needle tips spaced apart from each other at predetermined positions.

Preferably, each of the hyperthermia needles 6 further has a head portion 62 that is disposed on top of the substrate 3, and the needle portion 61 extends downwardly from the head portion 62 and through the substrate 3. The electromagnetic hyperthermia assembly further includes a clamp plate 4 that is disposed on top of the head portions 62 of the hyperthermia needles 6, and a plurality of fasteners 5 that fasten the clamp plate 4, the substrate 3 and the head portions 62 of the hyperthermia needles 6 together in a manner that the head portions 62 of the hyperthermia needles 6 are sandwiched between the clamp plate 4 and the substrate 3.

In this preferred embodiment, the substrate 3 of a rectangular shape is provided with four spaced-apart insertion holes 31 (only one is shown), four fastening holes 32, each of which is disposed between two corresponding adjacent ones of the insertion holes 31, and a central hole 33 that is surrounded by the insertion holes 31 and the fastening holes 32. There are in total five through holes 91 for the guiding plate 9 in this embodiment. Four of the through holes 91 are respectively aligned with the insertion holes 31 of the substrate 3.

The clamp plate 4 of a rectangular shape has a plurality of counter-sink holes 41 and a central hole 42 that is surrounded by the counter-sink holes 41. The fastening holes 32 are respectively aligned with the counter-sink holes 41, and the central hole 33 is aligned with the central hole 42.

Each of the hyperthermia needles 6 is made of a magnetically inducible metal material, such as a ferrous material. The needle portion 61 of each of the hyperthermia needles 6 extends downwardly through a respective one of the insertion holes 31 of the substrate 3.

Each of the fasteners 5 has a screw head 50 that is received in a respective one of the counter-sink holes 41, a screw rod 51 that extends downwardly from the screw head 50 through a respective one of the fastening holes 32 and that has a thread portion 53 projecting downwardly from the substrate 3, and a nut 52 that engages the thread portion 53. Fastening of the clamp plate 4 and the substrate 3 with the head portions 62 of the hyperthermia needles 6 being sandwiched therebetween by the fasteners 5 is achieved by means of a threaded engagement between the nut 52 and the thread portion 53 of each fasteners 5.

In this preferred embodiment, the numbers of the hyperthermia needles 6 and the fasten members 5 are both four. In actual implementation, the numbers of the hyperthermia needles 6, the fasten members 5, and the insertion holes 31 of the substrate 3 may vary. However, it is suggested that the number of the insertion holes 31 be at least two for insertion of two hyperthermia needles 6, respectively.

In this preferred embodiment, the electromagnetic hyperthermia assembly further includes a thermometric needle 7 that is attached to the substrate 3 and that extends downwardly through the substrate 3 and the guiding plate 9, and a thermometric detector 8 that is disposed inside the thermometric needle 7. Preferably, the thermometric needle 7 is surrounded by the hyperthermia needles 6 and has a receiving hole 70 that extends lengthwise therein for insertion of the thermometric detector 8. Preferably, the thermometric needle 7 further has a connection portion 72 that is disposed above the substrate 3, and a detecting needle portion 71 that projects downwardly from the connection portion 72 and that extends through the substrate 3. The receiving hole 70 extends from a top end of the connection portion 72 to a predetermined depth of the detecting needle portion 71. The connection portion 72 is disposed on top of the clamp plate 4. The detecting needle portion 71 extends downwardly through the central hole 42 of the clamp plate 4 and the central hole 33 of the substrate 3. More preferably, the thermometric needle 7 is made of a magnetic metal material, such as a ferrous material, and the thermometric detector 8 is a thermocouple.

Preferably, one of the through holes 91 of the guiding plate 9 permits insertion of the detecting needle portion 71 of the thermometric needle 7 thereinto. Since the others of the through holes 91 of the guiding plate 9 are respectively aligned with the insertion holes 31 of the substrate 3, the through holes 91 of guiding plate 9 keep the needle portions 61 of the hyperthermia needles 6 and the detecting needle portion 71 of the thermometric needle 7 spaced apart from each other. When the hyperthermia needles 6 and the thermometric needle 7 are inserted into the tissue or organ of the living body to be treated, the guiding plate 9 prevents any of the needle tips of the hyperthermia needles 6 and a needle tip of the thermometric needle 7 from becoming oblique, so that the distance between any two adjacent ones of the hyperthermia needles 6 and the thermometric needle 7 remains unchanged.

In actual use, the hyperthermia needles 6 are first secured to the substrate 3 and the clamp plate 4 with the head portions 62 clamped therebetween with the aid of the fasteners 5. Then, the guiding plate 9 is disposed below the substrate 3 with extensions of the hyperthermia needles 6 respectively through the through holes 91. By virtue of the guiding and retaining actions of the guiding plate 9, the needle portions 61 of the hyperthermia needles 6 are kept spaced apart from each other at the predetermined positions when the needle portions 61 are inserted into the tissue or organ to be treated. After the needle portions 61 are positioned, the thermometric needle 7 is inserted into the tissue or organ through the central hole 42 of the clamp plate 4, the central hole 33 of the substrate 3, and the corresponding through hole 91 of the guiding plate 9 such that the detecting needle portion 71 of the thermometric needle 7 is surrounded by the needle portions 61 of the hyperthermia needles 6. The thermometric detector 8 disposed inside the thermometric needle 7 is in signal connection with a coil controller (not shown).

The coil controller is able to apply a changeable magnetic field under a specified frequency. Consequently, the hyperthermia needles 6 and the thermometric needle 7 are induced to generate heat. The temperature of the tissue or organ to be treated is increased due to the heat generated by the hyperthermia needles 6 and the thermometric needle 7 so as to damage tumorous cells therein. The temperature of the tissue or organ subjected to the hyperthermia therapy is detected and monitored by the thermometric detector 8 so as to control the temperature of the tissue or organ through adjustment of the strength of the magnetic field. However, the thermometric needle 7 and the thermometric detector 8 may be omitted in other embodiments.

In addition to killing of the tumorous cells in the tissue or organ subjected to the hyperthermia therapy, the heat generated by the hyperthermia needles 6 is also able to burn the blood vessels located in the tissue or organ so as to stop bleeding of the tissue or organ and so as to avoid a massive hemorrhage and reduce surgery risk.

After the hyperthermia therapy is finished, the fasteners 5 are loosened, and the substrate 3, the hyperthermia needles 6, the clamp plate 4, the guiding plate 9, the thermometric needle 7 and the thermometric detector 8 are released from each other and can be independently cleaned and sterilized. By virtue of a separable design of the electromagnetic hyperthermia assembly, the constituent components of the electromagnetic hyperthermia assembly can be individually replaced and the electromagnetic hyperthermia assembly is unnecessary to be discarded in whole when only one or some of the constituent components is or are damaged. In addition, the hyperthermia needles 6 may have different lengths in accordance with the contour of the tissue or organ to be subjected to the hyperthermia therapy.

Alternatively, the screw heads 50 of the fasteners 5 may abut against the substrate 3 with the screw rods respectively extending upwardly through the substrate 3 and the clamp plate 4 from the screw heads 50 and the thread portions 53 respectively projecting upwardly from the clamp plate 4, while the nuts 52 are disposed above the clamp plate 4 and respectively engage the thread portions 53. Thereby, the clamp plate 4 and the substrate 3 likewise cooperatively clamp the head portion 62 of the hyperthermia needles 6 therebetween.

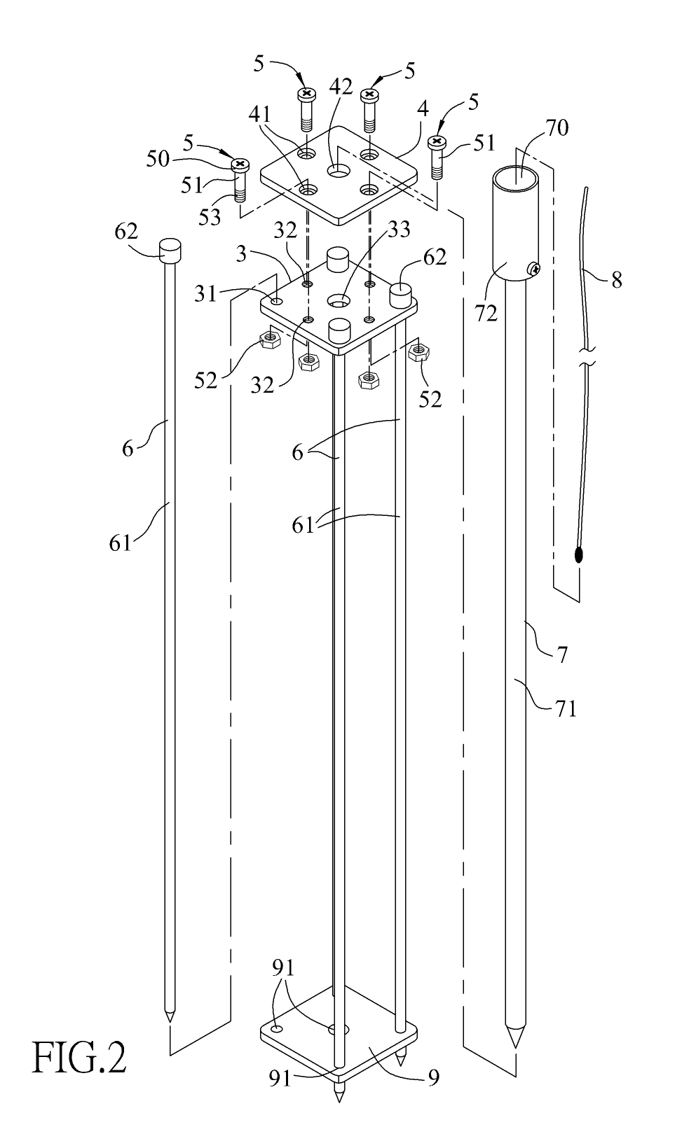

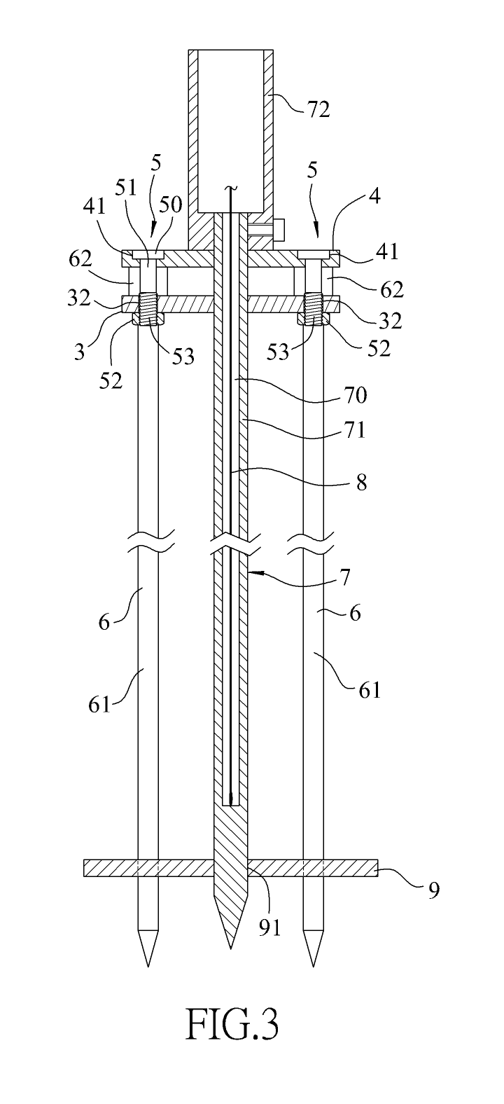

During actual implementation, in the arrangement of FIGS. 2 and 3, the nuts 52 of the fasteners 5 that are respectively engaged with the thread portions 53 may be embedded in the substrate 3, on in the clamp plate 4 in the alternative design.

Moreover, the fasteners 5 may be in other forms, such as clamps, snap-fit fasteners, or quick release fasteners. Since the feature of the electromagnetic hyperthermia assembly of this invention does not reside in the structure of the fasteners 5, further details of the same are omitted herein for the sake of brevity.

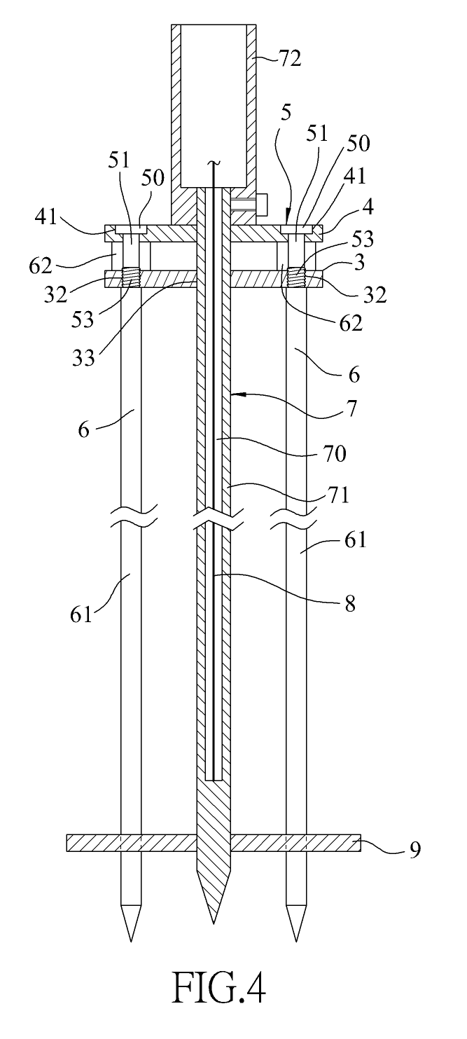

FIG. 4 illustrates the second preferred embodiment of an electromagnetic hyperthermia assembly according to the present invention, which has a structure generally similar to that of the first preferred embodiment. However, the nut 52 of each of the fasteners 5 is omitted. In this preferred embodiment, each of the fastening holes 32 has an inner threaded surface.

To lock the clamp plate 4 and the substrate 3 of the electromagnetic hyperthermia assembly, the screw rod 51 of each of the fasteners 5 is extended downwardly through the respective one of the counter-sink holes 41 of the clamp plate 4 such that the thread portion 53 of the screw rod 51 is engaged with the respective one of the fastening holes 32 of the substrate 3 until the head portions 62 of the hyperthermia needles 6 are sandwiched between the clamp plate 4 and the substrate 3. Alternatively, the counter-sinkholes 41 of the clamp plate 4 may be modified as thread holes, and the fastening holes 32 of the substrate 3 may be modified as counter-sink holes. In such case, the screw rod 51 of each of the fasteners 5 is extended upwardly through the respective one of the counter-sinkholes (not shown) of the substrate 3 such that the thread portion 53 of the screw rod 51 is engaged with the respective one of the thread holes (not shown) of the clamp plate 4.

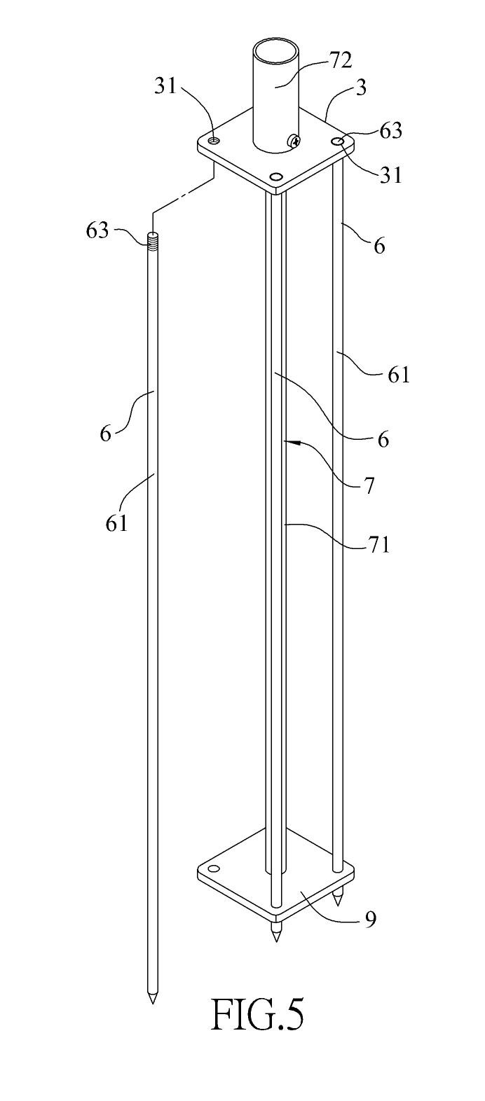

FIG. 5 illustrate the third preferred embodiment of an electromagnetic hyperthermia assembly according to the present invention, which has a structure generally similar to that of the first preferred embodiment. However, the clamp plate 4, the fasteners 5 and the head portion 62 (see FIG. 1) of each of the hyperthermia needles 6 are omitted. In this preferred embodiment, the insertion holes 31 of the substrate 3 are threaded holes. The needle portion 61 of each of the hyperthermia needles 6 has a screw thread segment 63 that is distal from the needle tip. When the substrate 3 is assembled with the hyperthermia needles 6, the screw thread segment 63 of each of the hyperthermia needles 6 is upwardly and threadedly engaged with a respective one of the insertion holes 31 of the substrate 3. By virtue of a threaded engagement between the substrate 3 and the hyperthermia needles 6, the electromagnetic hyperthermia assembly is likewise easily disassembled for convenient maintenance, cleaning and sterilization.

To sum up, by virtue of the structural arrangement of the electromagnetic hyperthermia assembly, the needle tips of the hyperthermia needles 6 are spaced apart from each other at predetermined positions by means of the guiding plate 9. The hyperthermia needles 6 are individually replaceable and may be individually dissembled for easy cleaning, maintenance and sterilization operations. The hyperthermia needles 6 may also come in different lengths or number so as to conform to the contour of the tissue or organ to be treated. In addition, by virtue of the thermometric needle 7 and the thermometric detector 8, the temperature of the tissue or organ subjected to the hyperthermia therapy is monitored and may be controlled.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. An electromagnetic hyperthermia assembly, comprising:
a substrate;
a plurality of spaced-apart hyperthermia needles respectively connected to said substrate, each of said hyperthermia needles having a needle portion that extends through and downwardly from said substrate and that has a needle tip distal from said substrate; and
a guiding plate disposed below said substrate and having a plurality of spaced-apart through holes,
wherein said needle portions of said hyperthermia needles respectively and removably extend through said through holes such that said guiding plate is movable along said needle portions and between said substrate and said needle tips of said needle portions, said through holes keeping said needle tips spaced apart from each other at predetermined positions.

2. The electromagnetic hyperthermia assembly as claimed in claim 1, wherein each of said hyperthermia needles further has a head portion disposed on top of said substrate, said needle portion extending downwardly from said head portion and removably through said substrate, said electromagnetic hyperthermia assembly further comprising a clamp plate disposed on top of said head portions of said hyperthermia needles, and a plurality of fasteners that removably fasten said clamp plate, said substrate and said head portions of said hyperthermia needles together in a manner that said head portions of said hyperthermia needles are sandwiched between said clamp plate and said substrate.

3. The electromagnetic hyperthermia assembly as claimed in claim 2, wherein said clamp plate has a plurality of counter-sink holes, said substrate having a plurality of fastening holes respectively aligned with said counter-sink holes.

4. The electromagnetic hyperthermia assembly as claimed in claim 3, wherein each of said fasteners has a screw head that is received in a respective one of said counter-sink holes, a screw rod that extends downwardly from said screw head through a respective one of said fastening holes and that has a thread portion projecting downwardly from said substrate, and a nut that engages said thread portion.

5. The electromagnetic hyperthermia assembly as claimed in claim 3, wherein each of said fastening holes is a screw hole, each of said fasteners having a screw head that is received in a respective one of said counter-sink holes, and a screw rod that extends downwardly from said screw head and that extends threadedly through said screw hole in said substrate.

6. The electromagnetic hyperthermia assembly as claimed in claim 1, further comprising a thermometric needle that is attached to said substrate and that extends downwardly through said substrate, and a thermometric detector that is disposed inside said thermometric needle.

7. The electromagnetic hyperthermia assembly as claimed in claim 6, wherein said thermometric needle has a receiving hole that extends lengthwise therein for insertion of said thermometric detector.

8. The electromagnetic hyperthermia assembly as claimed in claim 7, wherein said thermometric needle further has a connection portion that is disposed above said substrate, and a detecting needle portion that projects downwardly from said connection portion and that extends through said substrate.

9. The electromagnetic hyperthermia assembly as claimed in claim 8, wherein said receiving hole extends from a top end of said connection portion to a predetermined depth of said detecting needle portion.

10. The electromagnetic hyperthermia assembly as claimed in claim 8, wherein each of said hyperthermia needles further has a head portion disposed on top of said substrate, said needle portion extending downwardly from said head portion and through said substrate, said electromagnetic hyperthermia assembly further comprising a clamp plate disposed on top of said head portions of said hyperthermia needles, and a plurality of fasteners that removably fasten said clamp plate, said substrate and said head portions of said hyperthermia needles together in a manner that said head portions of said hyperthermia needles are sandwiched between said clamp plate and said substrate, said connection portion being disposed on top of said clamp plate, said detecting needle portion extending downwardly through said clamp plate and said substrate.

11. An electromagnetic hyperthermia assembly, comprising:
a substrate;
a plurality of spaced-apart hyperthermia needles that are respectively connected to said substrate, each of said hyperthermia needles having a needle portion that extends through and downwardly from said substrate and that has a needle tip distal from said substrate;
a thermometric needle that is attached to said substrate, said thermometric needle extending through and downwardly from said substrate; and
a thermometric detector that is disposed inside said thermometric needle.

12. The electromagnetic hyperthermia assembly as claimed in claim 11, wherein said thermometric needle is surrounded by said hyperthermia needles.

13. The electromagnetic hyperthermia assembly as claimed in claim 11, wherein said thermometric needle has a receiving hole that extends lengthwise therein for insertion of said thermometric detector.

14. The electromagnetic hyperthermia assembly as claimed in claim 13, wherein said thermometric needle further has a connection portion that is disposed above said substrate, and a detecting needle portion that projects downwardly from said connection portion and that extends through said substrate.

15. The electromagnetic hyperthermia assembly as claimed in claim 13, wherein said receiving hole extends from a top end of said connection portion and to a predetermined depth of said detecting needle portion.
